(19) United States
(12) Patent Application Publication
(10) Pub. No.: US 2021/0230313 A1
(43) Pub. Date: Jul. 29, 2021

(54) SULFUR-CONTAINING GLYCANS AND POLYSACCHARIDES AND METHODS OF CHEMOENZYMATIC SYNTHESIS THEREOF

(71) Applicants: The Board of Regents of the University of Oklahoma, Oklahoma City, OK (US); Rensselaer Polytechnic Institute, Troy, NY (US)

(72) Inventors: Paul L. DeAngelis, Edmond, OK (US); Dixy E, Green, Edmond, OK (US); Robert J. Linhardt, Albany, NY (US); Xing Zhang, Albany, NY (US); Victor Schuitz, Gloversville, NY (US)

(21) Appl. No.: 15/734,499

(22) PCT Filed: May 24, 2019
(86) PCT No.: PCT/US2019/033959

§ 371 Date: Dec. 2, 2020

Related U.S. Application Data
(60) Provisional application No. 62/680,363, filed on Jun. 4, 2018.

(51) Int. Cl. C08B 37/00; C12P 19/04; C12P 19/18; A61K 47/61; A61L 24/08; C08B 37/08

(52) U.S. Cl. CPC C08B 37/0075; C12P 19/04; C08B 37/0072; A61K 47/61; A61L 24/08; C12P 19/18

(57) ABSTRACT

Described herein are sulfur-containing glycosaminoglycans, and methods of making and using them. For example, heparosan and hyaluronan analogs are described which incorporate one or more sulfur-containing sugar units that include one or more free sulfhydryls and/or one or more thio-glycosidic linkages. In some embodiments, the glycosaminoglycans are included in pharmaceutical or bioadhesive compositions.

### RELATED APPLICATIONS

This application claims the benefit of U.S. Provisional Application No. 62/680,363, filed Jun. 4, 2018. The content of the aforementioned application is expressly incorporated herein by reference in its entirety.

### STATEMENT REGARDING FEDERALLY SPONSORED RESEARCH OR DEVELOPMENT

This invention was made with government support under Contract Number R01 HL062244-05A1 awarded by the National Institutes of Health. The government has certain rights in the invention.

### FIELD

Described herein are sulfur-containing (e.g., with thiol groups and/or thioglycosidic linkages) glycosaminoglycans such as heparosan and hyaluronan, and methods of making and using them. In some such embodiments, the glycosaminoglycans are included in pharmaceutical or bioadhesive compositions.

### BACKGROUND

Glycosaminoglycans (GAGs) are sugar polymers composed of repeating disaccharide units that include a hexosamine. Hyaluronan (hyaluronic acid; HA) and heparosan (HEP) are two examples of GAGs that are composed of the same two monosaccharides, but connected through different glycosidic linkages, which give each of the polysaccharides their unique biological effects. HA ([-4-GlcAβ1-3-GlcNAβ1-]ₙ) has multiple binding partners within the body, influencing the regulation of proliferation, cellular migration, and inflammation. HEP ([-4-GlcAβ1-4-GlcNAc-α1-]ₙ), the unmodified precursor backbone of heparan sulfate and heparin, on the other hand, has no other known function within the body to date. N-deacetylase/N-sulfotransferase initiates the transformation of HEP to heparan sulfate (HS), paving the way for further modifications by C₅-epimerase and O-sulfotransferases. These modifications create biologically active products, either HS or the more highly modified heparin. The differences in degree and pattern of sulfation result in a multitude of unique carbohydrate species with hundreds of binding partners within the body.

GAG synthases or polymerases are types of glycosyltransferases that polymerize the repeating disaccharide chains in various organisms including in animals and in certain pathogenic microbes that use similar polymers for extracellular capsules that camouflage against host defenses. The microbial enzymes, especially from the Gram-negative bacteria Pasteurella multocida, have very robust behavior in vitro and have been harnessed to make defined oligosaccharides, quasi-monodisperse polysaccharides, and analog-containing polymers. The Pasteurella enzymes are bifunctional glycosyltransferases that have two independent active sites that extend GAG chains with either a hexosamine or an uronic acid monosaccharide unit. Each site contains substrate-binding pockets for both the UDP-sugar donor and the oligosaccharide acceptor; the latter interacts with the nascent GAG chain. Other glycosyltransferases from Escherichia coli (e.g., KfiA and KfiC) employ two separate polypeptides to do the same task of GAG polymer assembly.

### SUMMARY

Some embodiments relate to a composition that includes an polymer comprising the repeat structure (-4-GlcA-1-beta-S-4-GlcNAc-1-alpha-)ₙ, or S-4-GlcNAc-1-alpha-(-4-GlcA-1-beta-O-4-GlcNAc-1-alpha-)ₙ, wherein n is a positive integer greater than or equal to 1; these polymers are heparosan-like species or sulfur-modified heparosan polymers. In some embodiments, the composition includes an polymer comprising the repeat structure (-4-GlcA-1-beta-O-3-GlcNAc(4S)-1-beta-)ₙ or GlcNAc(4S)-1-beta-(-4-GlcA-1-beta-O-3-GlcNAc-1-beta-)ₙ, wherein n is a positive integer greater than or equal to 1; these polymers are HA-like or hyaluronan-like species. The ‘O’ or the ‘S’ represents the linkage between the sugar units or sulfur-modified HA polymers.

Some embodiments relate to a method of chemoenzymatically producing a glycosaminoglycan polymer having a repeat structure that includes at least one sulfur-containing sugar unit. In some embodiments, the method includes: combining a recombinant glycosaminoglycan synthase with at least two UDP-sugars, wherein at least one of the UDP-sugars is a UDP-sulfur-containing sugar, whereby the recombinant glycosaminoglycan synthase polymerizes the at least two sugars to provide a glycosaminoglycan polymer having a repeat structure, and wherein the repeat structure comprises at least one sulfur-containing sugar.

In some embodiments, the method includes: combining a recombinant glycosaminoglycan synthase with a functional acceptor and at least two UDP-sugars, wherein at least one of the UDP-sugars is a UDP-sulfur-containing sugar, whereby the recombinant glycosaminoglycan synthase polymerizes the at least two sugars to provide a glycosaminoglycan polymer having a repeat structure, wherein the repeat structure comprises at least one sulfur-containing sugar.

In some embodiments, the method includes: combining a recombinant glycosaminoglycan synthase with a functional acceptor and one UDP-sulfur-containing sugar, whereby the recombinant glycosaminoglycan synthase provides a glycosaminoglycan polymer that comprises at least one sulfur-containing sugar. In some such cases, the polymer with a pendant sulfur-containing sugar unit is termed ‘end-labeled’ or ‘end-tagged.’

Some embodiments relate to a bioadhesive composition. In some embodiments, the bioadhesive composition includes a composition as described herein; a glycosaminoglycan polymer produced by a method described herein; and/or the glycosaminoglycan polymer-therapeutic agent conjugate produced by a method described herein.

Some embodiments relate to a pharmaceutical composition such as a pharmaceutical drug delivery composition. In some embodiments, the pharmaceutical drug delivery composition includes at least one conjugate comprising at least one therapeutic drug conjugated to at least one glycosaminoglycan polymer, wherein the glycosaminoglycan polymer is selected from the group consisting of: (a) a composition as described herein, and (b) a glycosaminoglycan polymer produced by the method as described herein; and a pharmaceutically acceptable carrier.

Some embodiments relate to a method, comprising: administering a therapeutically effective amount of the pharmaceutical drug delivery composition as described herein to a mammalian patient so as to induce a therapeutic effect in the mammalian patient and treat a disease or condition from which the mammalian patient suffers and/or reduce the occurrence of a disease or condition to which the mammalian patient is predisposed. In some embodiments, the therapeutically effective amount of the pharmaceutical drug delivery composition is injected into the mammalian patient.

Some embodiments relate to a composition, comprising: a polymer comprising the repeat structure (-4-GlcA-1-beta-S-4-GlcNAc-1-alpha-O)ₙ, wherein n is a positive integer greater than or equal to 1. Some embodiments relate to a composition, comprising: a polymer comprising the repeat structure (-4-GlcA-1-beta-O-4-GlcNAc-1-alpha-S)ₙ, wherein n is a positive integer greater than or equal to 1. Some embodiments relate to a composition, comprising: a polymer comprising the repeat structure (-4-GlcA-1-beta-S-4-GlcNAc-1-alpha-S)n, wherein n is a positive integer greater than or equal to 1. In some embodiments, any of the aforementioned polymers is an isolated polymer.

### BRIEF DESCRIPTION OF THE SEVERAL VIEWS OF THE DRAWINGS

FIG. 1 is a graphical depiction of one non-limiting example of a GAG synthesis method (C5 epi, C₅ epimerase; ST, sulfotransferase; UDP, uridine diphospho; PAPS, phosphoadenosine phosphosulfate; R, any functional group or moiety).

FIGS. 2A-2C are a non-limiting depictions of sulfur-tagged GAGs in accordance with some embodiments of the compositions and methods described herein.

FIG. 3 is a graphical depiction of one non-limiting depiction of the utility of sulfur-linked GAGs in accordance with some embodiments of the compositions and methods described herein. Uncleavable S-links will not be fragmented by and/or inhibit the action of mammalian heparanase thus reduce the progress of cancer.

FIG. 4 includes a flow-chart showing an example of a method of producing sulfur-containing GAGs, as well as some novel molecules created thereby.

FIG. 5 is a depiction of a chemical synthesis schema for production of UDP-4-thio sugars (4S) in accordance with one embodiment of the presently disclosed inventive concepts (the GlcNAc isomer shown, compound 11).

FIG. 6 includes mass spectra of sulfur-containing UDP-sugars produced in accordance with the presently disclosed inventive concept(s). The masses of the sulfur donor targets (622 Da) are highlighted in gray.

FIG. 7A is a spectra showing mass spectroscopic detection of UDP-4-thio-GlcNAc sugar incorporation by PmHS-G synthase; the S group adds on 16 extra Daltons of mass (i.e. the weight difference between natural abundance sulfur and oxygen atoms) to the GAG chain in comparison to the natural O group.

FIG. 7B includes two mass spectra that together show an analysis of heparosan single sugar extension reactions (top, 4S-GlcNAc; bottom, natural GlcNAc).

FIG. 8 is an image of a gel following PAGE that depicts polysaccharides made with either the two natural UDP-donors (4-OH) to form native HEP, or the thio-GlcNAc analog (4-SH) and UDP-GlcA donors to form Hemi-A HEP. The susceptibility of the two polymers to the bacterial heparin lyase (+lanes) is also probed.

FIG. 9 includes four panels of mass spectrometry data demonstrating detection of some S-tagged and S-linked heparosan embodiments. The top panel shows detection of S-GlcNAc-R (1 sugar added to acceptor terminus, R), the second panel shows detection of [S-GlcNAc-GlcA]_{1,2}-R (2 or 3 sulfur-sugars added, respectively), the third panel shows detection of O-GlcNAc-R, and the fourth panel shows detection of [O-GlcNAc-GlcA]_{1,2}-R, wherein R=Hep₃. The arrows denote major products of expected mass.

FIG. 10 includes two panels of mass spectra data in accordance with some embodiments.

FIG. 11 includes two images of agarose gels showing detection of some thio-tagged hyaluronan (HA) embodiments. The top image shows the stained polysaccharides (Stains-All panel), and the bottom image shows fluorescent antibody protein (Fluor panel) upon imaging with ultraviolet light. The ‘D’ debotes the antibody-linked dimer (HA-IgG-HA); the ‘M’ denotes a monomer HA chain.

### DETAILED DESCRIPTION

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The terms “heparosan,” “N-acetylheparosan,” and “unsulfated, unepimerized heparin” are used interchangeably herein and will be understood to refer to a carbohydrate chain with a repeat structure of ([-4-N-acetylglucosamine-α1,4-glucuronic acid-β1-]ₙ), wherein n is 1 or greater. In certain non-limiting examples, n may be from about 2 to about 5,000.

The terms “hyaluronan,” “hyaluronic acid,” and “HA” are used herein interchangeably and will be understood to refer to a carbohydrate chain with a repeat structure of ([-3-N-acetylglucosamine-α1,4-glucuronic acid-β1-]ₙ), wherein n is 1 or greater. In certain non-limiting examples, n may be from about 2 to about 5,000.

The term “oligosaccharide” generally denotes n (i.e., the number of disaccharide units) as being from about 1 to about 11, while the term “polysaccharide” denotes n as being equal to or greater than 12.

The term “unnatural glycosaminoglycan” (i.e., unnatural GAG) as used herein refers to a composition of matter not normally found in known living vertebrates, animals or microbes; different arrangements and/or structures of chemical groups are added by the hand of man.

The term “chimeric glycosaminoglycan” (i.e., chimeric GAG) as used herein refers to a composition of matter not normally found in known living vertebrates, animals, or microbes; different arrangements of GAG structures or segments are added by the hand of man.

The terms “unnatural sugar” and “sugar analog” are used herein interchangeably, and will be understood to refer to a sugar analog that is not found in mammals in a native state, that is, a sugar analog that is produced by the hand of man. This sugar unit may be a component of a precursor UDP-sugar, or an acceptor, or the monosaccharide itself.

The term “UDP-sugar” as used herein refers to a carbohydrate modified with uridine diphosphate (e.g., UDP-N-acetylglucosamine, UDP-GlcNAc, etc.).

The term “functional acceptor” as used herein refers to a primer for accepting the incoming donor sugar transferred by the synthase enzyme. This acceptor may also contain other non-sugar groups or derivatives for uses not limited to either containing directly and/or facilitating the subsequent addition of chemical, biological or therapeutic moieties that confer or enhance action(s) of the final GAG polymer molecule.

The term “analog” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all; in the latter case, these drugs can often act as bait or blockers of activity. Alternatively and/or in addition thereto, for a chemical, an analog may be any structure that has the functionalities (including alterations or substitutions in the core moiety) desired, even if comprised of different atoms or isomeric arrangements.

As used herein, the term “active agent(s),” “active ingredient(s),” “pharmaceutical ingredient(s),” “therapeutic,” “medicant,” “medicine,” “biologically active compound,” and “bioactive agent(s)” are defined as drugs and/or pharmaceutically active ingredients.

The term “pharmaceutically acceptable” refers to compounds and compositions that are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule/composition can be biologically active through its own functionalities, or may be biologically active based on its ability to activate, modulate, or inhibit molecules/compositions having their own biological activity. In addition, biological activity observed in in vitro proxy models is indicative of in vivo action of a molecule/composition.

Certain abbreviations used within the context of this disclosure include, but are not limited to: AMAC, 2-aminoacridone; ANTS, 8-aminonaphthalene-1,3,6-trisulfonic acid; Glc, glucose; GlcA, glucuronic acid; GlcNAc, N-acetylglucosamine; HA, hyaluronan; HEP, heparosan; Heparinase III, Heparin lyase III; MALDI-ToF MS, Matrix-Assisted Laser Desorption/Ionization Time-of-Flight Mass Spectrometry; PmHAS, P. multocida hyaluronan synthase; PmHS, P. multocida HEP synthase.

The entire contents of the below-referenced patents and patent applications are hereby expressly incorporated herein by reference: U.S. Ser. No. 14/937,369, filed Nov. 10, 2015; U.S. Ser. No. 13/325,181, filed Dec. 14, 2011, now U.S. Pat. No. 9,200,024, issued Dec. 1, 2015; U.S. Ser. No. 11/906,704, filed Oct. 3, 2007, now U.S. Pat. No. 8,088,604, issued Jan. 3, 2012; U.S. Ser. No. 60/849,034, filed Oct. 3, 2006; U.S. Ser. No. 11/651,379, filed Jan. 9, 2007, now U.S. Pat. No. 7,579,173, issued Aug. 25, 2009; U.S. Ser. No. 10/642,248, filed Aug. 15, 2003, now U.S. Pat. No. 7,223,571, issued May 29, 2007; U.S. Ser. No. 60/404,356, filed Aug. 16, 2002; U.S. Ser. No. 60/479,432, filed Jun. 18, 2003; U.S. Ser. No. 60/491,362, filed Jul. 31, 2003; U.S. Ser. No. 11/975,811, filed Oct. 22, 2007, now U.S. Pat. No. 7,771,981, issued Aug. 10, 2010; and U.S. Ser. No. 10/142,143, filed May 8, 2002, now U.S. Pat. Nos. 7,307,159; 6,444,447; 7,060,469; 7,741,091; 7,223,571; 6,951,743; 7,094,581; 8,735,102; 6,833,264; 6,987,023; 7,232,684; and 7,811,806.

Turning now to the presently disclosed embodiments, certain non-limiting aspects thereof are directed to compositions that include an isolated polymer comprising the repeat structure structure (-4-GlcA-1-beta-S-4-GlcNAc-1-alpha-O)ₙ or (-4-GlcA-1-beta-O-3-GlcNAc(4S)-1-beta-O)ₙ, or other positional isomers wherein n is at least 1. Structures of novel sulfur-containing analogs and native glycosaminoglycan structures are shown for comparison in Table 1. The final GAG polymer structure is dictated by the exact UDP-sugar analog donors (e.g., UDP-GlcNAc(4S), UDP-GlcA(4S). or other thiol-containing sugar donors) used in the synthesis. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. For example, some embodiments include a sulfur-containing sugar polymer having 12 to 15 monomers in the polymer. In some embodiments, n is greater than 50. In some embodiments, n is between 5 and 20. Some embodiments include a plurality of sulfur-containing sugar polymers as described herein, each having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 monomers in the polymer. In some embodiments, the average number of monomers of the polymers in the plurality of sugar polymers is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the average number of monomers of the polymers in the plurality of sugar polymers is between 12 and 15.

TABLE 1

| | |
|---|---|
| Native Heparosan | (-4-GlcA-1-beta-O-4- |
| | GlcNAc-1-alpha-O)ₙ |
| Sulfur-containing | (-4-GlcA-1-beta-S-4- |
| Heparosan Analog 1 (Hemi-A) | GlcNAc-1-alpha-O)ₙ |
| Sulfur-containing | (-4-GlcA-1-beta-O-4- |
| Heparosan Analog 2 (Hemi-B) | GlcNAc-1-alpha-S)ₙ |
| Sulfur-containing | (-4-GlcA-1-beta-S-4- |
| Heparosan Analog 3 | GlcNAc-1-alpha-S)ₙ |
| (Complete S-links) | |
| Native Hyaluronic Acid (HA) | (-4-GlcA-1-beta-O-3- |
| | GlcNAc-1-beta-O)ₙ |
| Sulfur-containing HA Analog | (-4-GlcA-1-beta-O-3- |
| | GlcNAc(4S)-1-beta-)ₙ |
| Sulfur-containing | GlcNAc(4S)-1-beta-(-4-GlcA- |
| End-labeled HA Analog | 1-beta-O-3-GlcNAc-1-beta-)ₙ |
| Sulfur-containing | S-4-GlcNAc-1-alpha-(-4-GlcA- |
| End-labeled Heparosan Analog | 1-beta-O-4-GlcNAc-1-O-alpha-)ₙ |
| (e.g., one of many | |
| possible positional analogs) | |

In certain non-limiting embodiments, n may be greater than or equal to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10000, as well as any value therebetween. In certain other non-limiting embodiments, n is in a range having a lower value selected from the group consisting of about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, and about 100, as well as any value therebetween. The range may also have an upper value selected from the group consisting of about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, and about 10000, as well as any value therebetween. All value ranges resulting from combinations of the various upper and lower values indicated above fall within the scope of the presently disclosed embodiments. However, the above values are only provided as illustrative examples; an isolated polymer comprising one of the repeat structures above wherein n is greater than 10,000, or wherein n is in a range that falls outside the particular values listed above (such as, but not limited to, a range that has a lower value greater than 100 and/or an upper value greater than 10,000) also falls within the scope of the presently disclosed embodiments.

In some embodiments, at least a portion of the polymer is sulfated. In some embodiments, at least a portion of the polymer is epimerized, if desired. Alternatively, the polymer may be unsulfated and/or unepimerized. In addition, the polymer may be substantially monodisperse in size.

The term “substantially monodisperse in size” as used herein will be understood to refer to defined glycoasminoglycan polymers that have a very narrow size distribution, such as (but not limited to) a polydispersity value in a range of from about 1.0 to about 1.5. For example, substantially monodisperse glycosaminoglycan polymers having a molecular weight in a range of from about 3.5 kDa to about 0.5 MDa will have a polydispersity value (i.e., Mw/Mn, where Mw is the average molecular weight and Mn is the number average molecular weight) in a range of from about 1.0 to about 1.1, such as (but not limited to) in a range from about 1.0 to about 1.05. In yet another example, substantially monodisperse glycosaminoglycan polymers having a molecular weight in a range of from about 0.5 MDa to about 4.5 MDa will have a polydispersity value in a range of from about 1.0 to about 1.5, such as (but not limited to) in a range from about 1.0 to about 1.2.

Certain non-limiting embodiments of the present disclosure relate to a method for chemoenzymatically producing a glycosaminoglycan polymer having a repeat structure comprising at least one sulfur-containing sugar unit. In the method, a recombinant glycosaminoglycan synthase is combined with at least two UDP-sugars (in the presence or absence of a functional acceptor); at least one of the UDP-sugars is a UDP-sulfur-containing sugar. The recombinant glycosaminoglycan synthase polymerizes the at least two sugars to provide a glycosaminoglycan polymer having a repeat structure, wherein the repeat structure comprises at least one sulfur-containing sugar.

In certain particular (but non-limiting) embodiments, the glycosaminoglycan polymer so produced comprises (1) the repeat structure (-4-GlcA-1-beta-S-4-GlcNAc-1-alpha-)ₙ, wherein n is a positive integer greater than or equal to 1; or (2) the repeat structure (-4-GlcA-1-beta-O-3-GlcNAc(4S)-1-beta-)ₙ, wherein n is a positive integer greater than or equal to 1.

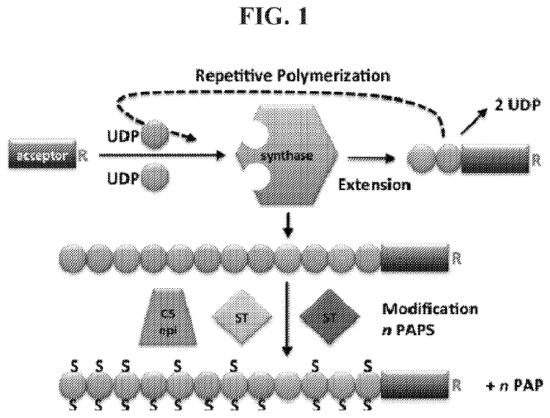

FIG. 1 includes a non-limiting example of a GAG synthesis method that includes chemoenzymatic HS synthesis processes. The bifunctional heparosan synthase (hexagon with two ‘empty’ circular active sites) can extend an acceptor with a reducing end (R) with sugar units (circles) from UDP-sugar donors (natural or analog) in a stepwise or a repetitive fashion (dashed arrow) to create a sugar polymer backbone. In some embodiments, this backbone is then modified with any of various combinations of HS biosynthetic enzymes (such as epimerase, C5 epi; or N- and O-sulfotransfereases, ST) to create epimerized, sulfated HS chains. The addition of the sulfate groups allows the binding of various heparin-active proteins to the HS. The sulfation position on the sugar ring as well as the local pattern on 2 to 20 sugar units, and the overall density of sulfates along a sugar chain help confer the binding specificity.

In certain embodiments, chain polymerization occurs after de novo initiation (i.e., without a functional acceptor). Alternatively, a functional acceptor may be combined with the glycosaminoglycan synthase and the at least two UDP sugars.

In certain embodiments, the UDP-sulfur-containing sugar is 4SH-GlcNAc or 4SH-GalNAc, or comprises a monosaccharide such as 4SH-GlcNAc or 4SH-GalNAc.

Any recombinant glycosaminoglycan synthase described or incorporated by reference herein may be utilized in the methods of the presently disclosed embodiments. For example, the recombinant glycosaminoglycan synthase utilized in accordance with the presently disclosed embodiments may be a recombinant hyaluronan synthase, a recombinant heparosan synthase, a recombinant chondroitin synthase, or any active fragment or mutant or chimeric construct thereof. The recombinant glycosaminoglycan transferase may be capable of adding only one UDP-sugar described herein above or may be capable of adding two or more UDP-sugars described herein above.

In one embodiment, the at least one recombinant glycosaminoglycan synthase is selected from the group consisting of (1) a recombinant hyaluronan synthase or active fragment or variant thereof, (2) a recombinant heparosan synthase or active fragment or variant thereof, (3) a recombinant chondroitin synthase or active fragment or variant thereof, and combinations thereof. In another embodiment, the at least one recombinant glycosaminoglycan synthase comprises a recombinant single action glycosyltransferase capable of adding only one of GlcA, GlcNAc, Glc, or a structural variant or derivative thereof. In yet another embodiment, the at least one recombinant glycosaminoglycan synthase comprises a recombinant synthetic chimeric glycosaminoglycan synthase capable of adding two or more of GlcA, GlcNAc, Glc, and a structural variant or derivative thereof.

Examples of recombinant hyaluronan synthases, active fragments and variants thereof, single action glycosyltransferases therefrom, and chimeric glycosaminoglycan synthases that include portions thereof and that may be utilized in accordance with the presently disclosed embodiments include, but are not limited to, those disclosed in U.S. Pat. No. 6,444,447, issued Sep. 3, 2002; U.S. Pat. No. 7,060,469, issued Jun. 13, 2006; U.S. Pat. No. 7,575,904, issued Aug. 18, 2009; U.S. Pat. No. 7,534,589, issued May 19, 2009; U.S. Pat. No. 7,741,091, issued Jun. 22, 2010; and U.S. Pat. No. 7,223,571, issued May 29, 2007. The entire contents of said patents are expressly incorporated herein by reference.

Examples of recombinant heparosan synthases, active fragments and variants thereof, single action glycosyltransferases therefrom, and chimeric glycosaminoglycan synthases that include portions thereof and that may be utilized in accordance with the presently disclosed embodiments include, but are not limited to, those disclosed in U.S. Pat. No. 7,307,159, issued Dec. 11, 2007; U.S. Pat. No. 7,771,981, issued Aug. 10, 2010; U.S. Pat. No. 8,088,604, issued Jan. 3, 2012; and U.S. Pat. No. 9,200,024, issued Dec. 1, 2015. The entire contents of said patents are expressly incorporated herein by reference.

When a functional acceptor is present, the UDP-sulfur-containing sugar may be provided in a stoichiometric ratio to the at least one functional acceptor such that the recombinant glycosaminoglycan synthase elongates the at least one functional acceptor to provide a polymer that is substantially monodisperse in size. For example (but not by way of limitation), the polymer may have a polydispersity value in a range of from about 1.0 to about 1.5.

In certain non-limiting embodiments, the functional acceptor may include at least one sugar unit, and wherein the sugar unit comprises at least one of uronic acid, a uronic acid analog comprising a substitution at at least one of the C2, C3, and C6 positions thereof, a hexosamine, and a hexosamine analog comprising a substitution at at least one of the C2, C3, and C6 positions thereof. Alternatively, the functional acceptor may include at least two sugar units, and wherein at least one of the at least two sugar units is selected from the groups above. The uronic acid may be further defined as a uronic acid selected from the group consisting of GlcA, iduronic acid (IdoA), GalUA, and structural variants or derivatives thereof. The hexosamine may be further defined as a hexosamine selected from the group consisting of GlcNAc, GalNAc, GlcN, GalN, and structural variants or derivatives thereof. The hexosamine analog may be further defined as a hexosamine analog selected from the group consisting of GlcN, GlcNAcNAc, GlcN[TFA], GlcNBut, GlcNPro, and 6-F-6-deoxyGlcNAc.

Another functional acceptor class that may be utilized in accordance with the one embodiment includes synthetic glycosides (i.e., sugars that have a non-sugar component at the reducing end) or similar synthetic carbohydrates. The synthetic portion substitutes for one of the natural sugar units; these molecules are less expensive and can possess useful groups.

In certain non-limiting embodiments, the glycosaminoglycan synthase and UDP sugars (as well as the at least one functional acceptor, if present) are combined in the presence of at least one divalent metal ion and in a buffer having a pH from about 4 to about 9. For example (but not by way of limitation), the divalent metal ion may be manganese, magnesium, cobalt, or nickel.

In one embodiment, the functional acceptor may include at least one of: an HA oligosaccharide, polysaccharide, or polymer; a chondroitin oligosaccharide, polysaccharide, or polymer; a chondroitin sulfate oligosaccharide, polysaccharide, or polymer; a heparosan oligosaccharide, polysaccharide, or polymer; a heparin oligosaccharide, polysaccharide, or polymer; a heparan oligosaccharide, polysaccharide, or polymer; an acceptor comprising a glycoside of uronic acid; a sulfated or modified oligosaccharide, polysaccharide, or polymer; and an extended acceptor selected from the group consisting of HA chains, chondroitin chains, heparosan chains, mixed glycosaminoglycan chains, analog containing chains, and combinations thereof.

In particular non-limiting embodiments, the at least one functional acceptor is GlcA-terminated or GlcNAc-terminated.

In certain non-limiting embodiments, the at least one functional acceptor may further include a moiety selected from the group consisting of a fluorescent tag, a radioactive tag, an affinity tag, a detection probe, a medicant, a biologically active agent, a therapeutic agent, and combinations thereof.

In certain non-limiting embodiments, at least one of the at least two UDP-sugars is radioactive or nuclear magnetic resonance-active; said radioactive or nuclear magnetic resonance-active UDP-sugar may also be the sulfur-containing UDP-sugar, or the non-sulfur-containing UDP sugar.

The method may further include the step(s) of sulfating (for example, chemically and/or enzymatically) and/or epimerizing at least a portion of the polymer. Alternatively, the polymer produced by the method may be unsulfated and unepimerized.

In certain particular (but non-limiting) embodiments, the functional acceptor comprises a therapeutic agent; as such, a method of chemoenzymatically producing a glycosaminoglycan polymer-therapeutic agent conjugate is also provided.

Other embodiments of the present disclosure also include bioadhesive compositions containing any of the sulfur-containing compositions (or conjugates containing same) disclosed or otherwise contemplated herein.

Other embodiments of the present disclosure also include pharmaceutical drug delivery compositions that contain at least one conjugate and a pharmaceutically acceptable carrier. The conjugate comprises at least one therapeutic drug conjugated to any of the sulfur-containing compositions disclosed or otherwise contemplated herein. For example (but not by way of limitation), the pharmaceutical drug delivery composition may be a sterile pharmaceutical formulation in a unit dosage format.

In certain non-limiting embodiments, the at least one therapeutic drug remains active following conjugation to the at least one glycosaminoglycan polymer. In certain non-limiting embodiments, the at least one therapeutic drug does not increase immunoreactivity of the at least one glycosaminoglycan polymer. In certain non-limiting embodiments, the at least one therapeutic drug is not an adjuvant.

In certain non-limiting embodiments, the conjugate may comprise a single glycosaminoglycan polymer conjugated to the at least one therapeutic drug. Alternatively, the conjugate may comprise a plurality of glycosaminoglycan polymers conjugated to the at least one therapeutic drug.

In certain non-limiting embodiments, the at least one glycosaminoglycan polymer comprises an activated group thereon to effect the conjugation to the at least one therapeutic drug, and wherein the reactive group is selected from the group consisting of (or consisting of) an aldehyde, alkyne, ketone, maleimide, thiol, azide, amino, carbonyl, sulfhydryl, hydrazide, phosphate, sulfate, nitrate, carbonate, ester, squarate, chelator, and combinations thereof.

In certain non-limiting embodiments, the at least one glycosaminoglycan polymer and the at least one therapeutic drug are covalently conjugated. Alternatively, the at least one glycosaminoglycan polymer and the at least one therapeutic drug may be non-covalently conjugated.

The at least one therapeutic drug may be any drug known or otherwise contemplated in the art that is capable of conjugation to a glycosaminoglycan polymer. For example but not by way of limitation, the therapeutic drug may be selected from the group consisting of a chemotherapy agent, an antineoplastic agent, a steroid, an antibiotic, an anti-inflammatory agent, an agent that has an action on a central nervous system of the mammalian patient, an antihistaminic, an antiallergic agent, an antipyretic, a respiratory agent, an antimicrobial agent, an antihypertensive agent, a calcium antagonist, an antipsychotic, an agent for Parkinson's disease, a vitamin, an antitumor agent, a cholinergic agonist, a mydriatic, an antidepressant agent, an antidiabetic drug, an anorectic agent, an antimalarial agent, a polypeptide therapeutic, a cytokine, a hormone, an enzyme, an antibody, an antibody fragment, an antiulcerative agent, an anticancer agent, a vaccine antigen, a polynucleotide, a nutrient, a small molecule, and combinations thereof.

Certain non-limiting embodiments of the present disclosure are related to a method that comprises administering a therapeutically effective amount of any of the pharmaceutical drug delivery compositions disclosed or otherwise contemplated herein to a mammalian patient so as to induce a therapeutic effect in the mammalian patient and treat a disease or condition from which the mammalian patient suffers and/or reduce the occurrence of a disease or condition to which the mammalian patient is predisposed.

In certain particular (but non-limiting) embodiments, the therapeutically effective amount of the pharmaceutical drug delivery composition is injected into the mammalian patient.

Further disclosure related to the pharmaceutical drug delivery compositions and conjugates utilized therein can be found in US Patent Application Publication No. 2010/0036001, published Feb. 11, 2010, the entire contents of which are hereby expressly incorporated herein by reference. Additional embodiments related to therapeutic agents, conjugation techniques, etc. disclosed therein also fall within the scope of the presently disclosed inventive concept(s).

The presently disclosed embodiments encompass methods of producing a variety of unique biocompatible molecules and coatings based on polysaccharides. The presently disclosed embodiments incorporate the propensity of certain recombinant enzymes, when prepared in a virgin state, to utilize various acceptor molecules as the seed for further polymer growth: naturally occurring forms of the enzyme or existing living wild-type host organisms do not display this ability. Thus, the presently disclosed embodiments allow for the addition of new chemical groups and thus forming unnatural glycosaminoglycan polymers that may facilitate coupling to other molecules or surfaces, even cells.

Some embodiments include a bioadhesive material including a composition as described herein such as a sulfur-containing GAG. Some embodiments include a method of making a bioadhesive material by providing, incorporating or including a composition as described herein such as a sulfur-containing GAG. Some embodiments include bioadhesive controlled drug delivery to, for example, localize a delivery device within the body to enhance the drug absorption process in a site-specific manner. Bioadhesion may be affected by the synergistic action of the biological environment, the properties of the polymeric controlled release device, and the presence of the drug itself. The delivery site and the device design may be dictated by the drug's molecular structure and its pharmacological behavior.

In the presently disclosed embodiments, the compositions disclosed or otherwise contemplated herein would be the natural substitute for poly(acrylic-acid) based materials. These GAGs are negatively-charged polymers as is poly(acrylic-acid), but glycosaminoglycans are naturally occurring molecules in the vertebrate body and would not invoke an immune response like a poly(acrylic-acid) material.

A second ingredient of this embodiment is a bioadhesive comprising an amount of any of the polymer compositions disclosed or otherwise contemplated herein directly polymerized onto a molecule with the desired pharmacological property or a polymer chain polymerized onto a matrix or liposome which in turn contains or binds the medicament.

The various polymers produced by the methods of the presently disclosed embodiments may be materials for incorporation, either directly or indirectly, into a scaffold for cell growth and implantation. In addition, the polymers may be attached to surfaces or devices via acceptor moiety or a direct chain interaction.

Some embodiments include skin substitutes or skin grafts, such as cultured epidermal autografts can provide permanent coverage of large area from a skin biopsy.

The various polymers produced by the methods of the presently disclosed embodiments may be incorporated, either directly or indirectly, onto cell surfaces. The polymers may be attached to cell surfaces or devices via acceptor moiety (for example, but not by way of limitation, a lipid conjugate).

The heparosan-containing polymers of the presently disclosed embodiments are also a starting material for sulfur-containing heparin-based anticoagulants, antivirals, proliferation modulators, anti-cancer agents (e.g., heparanase inhibitors), anti-inflammatory drugs, etc. Size defined molecules as well as analogs should allow a multitude of therapeutics to be created with potential for enhanced activity and better control.

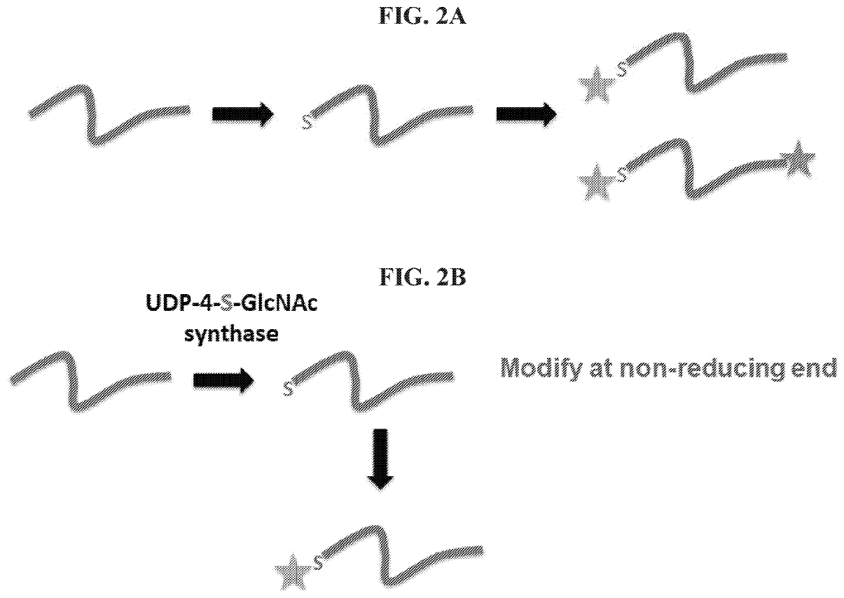

Some embodiments relate to a GAG comprising a sulfur molecule; here the sulfur is initially in the form of a free sulfhydryl group (—SH, thiol group). Some such embodiments are used as a handle to bind a moiety to the GAG, or to convert the GAG into a pharmaceutical composition. For example, FIG. 2A and FIG. 2B show some examples of sulfur-tagged GAGs that may be used as handles for drug delivery and/or conjugation. In some such embodiments include adding a drug or payload at a unique defined position to the GAG, and/or creating linker polymers with, for example, two orthogonal coupling sites. FIG. 2C includes some examples of sulfur-tagged GAGs being used as handles for drug delivery and/or conjugation.

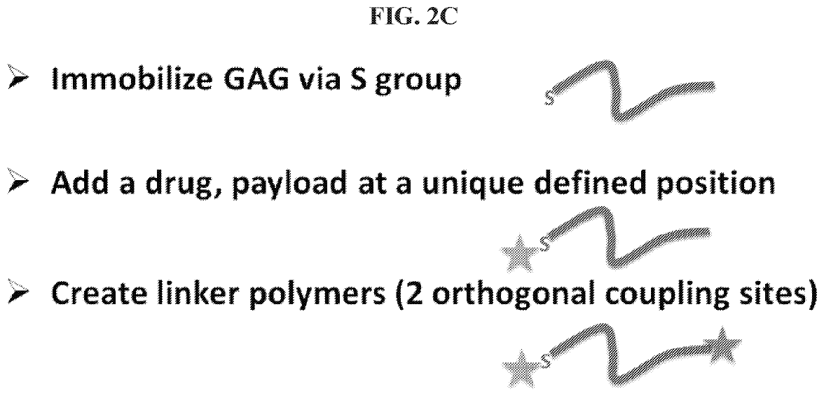

Some embodiments include sulfur-linked GAGs such as one containing a thioglycosidic linkage between sugar units, or X-S-X′, wherein X, X′=monosaccharide unit, or as shown in FIG. 3. In some embodiments, the sulfur-linked GAGs are used as drugs or for drug-binding. For example, sulfur-linked GAGs may be used as heparanase inhibitors and/or traps for oncology.

Heparanase is expressed by metastatic tumor cells and activated cells of the immune system may release FGF eliciting an indirect neovascular response. Heparanase acts as a regulatory switch mediating the release of specifically tailored saccharide structures within HS with restricted binding specificities. The actions of heparanase are linked to numerous human diseases, such as cancer, diabetes, and Alzheimer's.

Some embodiments include a heparanase inhibitor comprising a sulfur-containing GAG as described herein. In FIG. 3, one scenario is depicted where natural HS (shown as a long chain at the top-left of the figure) is digested or cleaved by heparanase forming HS fragments (shown as a fragmented chain in the upper-right part of the figure) that are are bioactive themselves or too short to bind growth factors; the cancer cell benefits from the HS fragments and/or newly available growth factors. In contrast, an artificial S-linked HS polymer (shown as a long chain in the lower left part of the figure) is not digestible by the heparanase, and may compete for endogenous HS, thus cancer does not receive signals that cause disease progression and tumor growth. Some embodiments include a method of treating, inhibiting, or preventing a disease in a subject in which heparanase expression is increased compared to a healthy control subject, comprising administering a a sulfur-containing GAG as described herein to the subject in which heparanase is expression is increased. Examples of diseases that may be treated, inhibited, or prevented by such a method include cancer, type I and type II diabetes, and Alzheimer's.

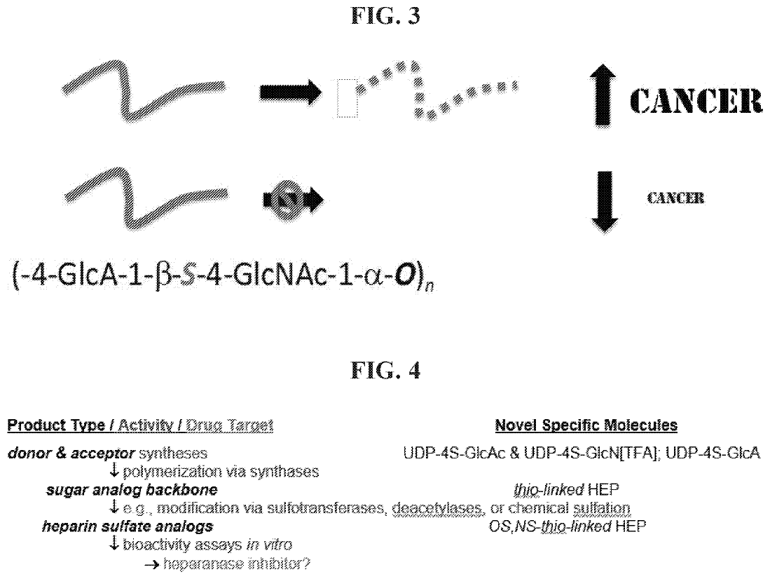

Some embodiments of the methods and compositions described herein include chemoenzymatic synthesis with UDP-sugars, UDP-sugar analogs, acceptors, and the heparosan synthases and then PAPS, epimerases, and sulfotransferases to form HS analogs. Some non-limiting examples are shown in FIGS. 1 and 4.

### EXAMPLES

### Example 1—Thio-Sugar Donor Synthesis and Use

Examples are provided herein below. However, the presently disclosed embodiments are to be understood to not be limited in its application to the specific experimentation, results and laboratory procedures. Rather, the Examples are simply provided as one of various embodiments and are meant to be exemplary, not exhaustive.

### Methods Related to Sulfur-Containing UDP-Sugar Syntheses

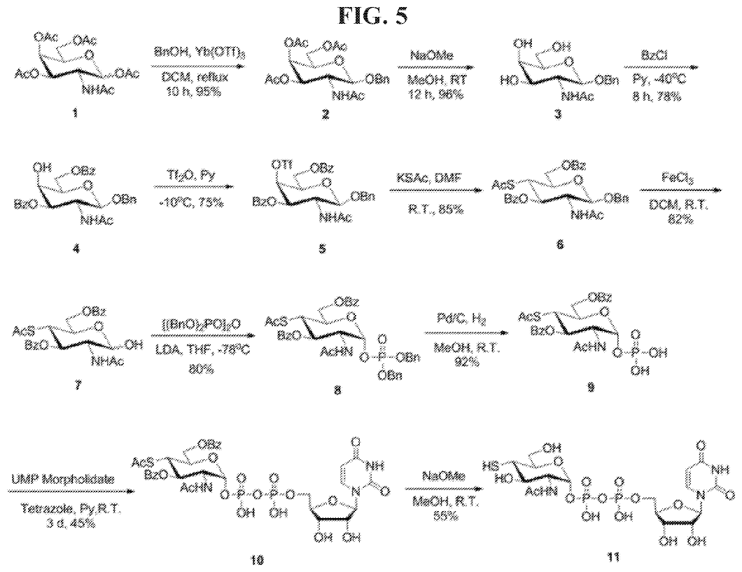

In this example, the first step in producing sulfur-containing glycans and polysaccharides is the production of UDP-4-thio sugars (4S) that serve as the donor of the sugar analog. FIG. 5 illustrates one non-limiting embodiment of a chemical synthesis schema for production of UDP-4SH-GlcNAc; however, it is to be understood that, while only one stereochemical series is shown, both Gal and Glc series were synthesized by similar processes as shown in FIG. 5. In addition, other sugar ring positions of the donor may be modified to add the sulfur group to other potential sites on the GAG chain. Alternatively, the other sugar unit of many GAGs, GlcA, may be modified with a thiol group and be employed in synthase-catalyzed extension reactions.

As shown in FIG. 5, synthesis in accordance with one embodiment started from the commercial available compound D-glucosamine pentaacetate 1. Refluxing 1 with benzyl alcohol and Yb(OTf)₃ in dichloromethane provided the O-benzyl glycoside of D-galactosamine/glucosamine tetraacetate 2 in good yields (Glc 95% and Gal 97%) and with excellent selectivity. Zemplen deacetylation reaction with sodium methoxide in methanol afforded triol 3 in quantitative yield. The placement of the C-4 thiol functionality was initiated through the reaction of Tf₂O in pyridine with 4 to afford the triflate 5. Nucleophilic displacement of triflate groups in 5 with potassium thioacetate in dimethylformamide afforded 4-SAc-GlcNAc derivatives 6 in 85-90% yields with the inversion of the C-4 configuration. The anomeric benzyl group was oxidatively removed affording 7, followed by phosphorylation with tetrabenzyl pyrophosphate to obtain the phosphorylated intermediate 8 in good yield (80%-84%) and with excellent α-selectivity. Deprotection of benzyl groups was achieved under hydrogenation to obtain the target benzoate-protected substrate 9 in good yields (85%-90%). The protected 4-SAc-N-acetylglucosamine-1-phosphate 9 was converted to the pyridinium salts and then stirred with UMP-morpholidate and tetrazole in pyridine for 3 days to afford the benzoate-protected UDP sugar. By treating the reaction crude with (Na⁺) resin, all the phosphate salts were converted to their sodium form and purified on a Biogel P2 column afforded the protected UDP derivative 10 in modest yields (45%-58%). Deprotection of 8 was carried out under standard Zemplen conditions followed by treatment of dithiothreitol (DTT) to cleave disulfide, affording the corresponding donors UDP-4-SH-GlcNAc 11.

### Materials and Methods Relating to Matrix-Assisted Laser Desorption/Ionization Time-Of-Flight Mass Spectrometry (MALDI-ToF MS)

UDP-sugars (or as shown later, reaction mixtures post-incubation) were co-spotted with an equal volume of 6.5 mg/mL 6-aza-2-thyothymine (ATT) matrix in 50% acetonitrile with 0.1% trifluoroacetic acid and air dried. An UltraFlex II MALDI instrument (Bruker, Billerica, Mass.) was used in negative ion mode with laser at 20% power, reflector, and 40% detector gain to obtain spectra.

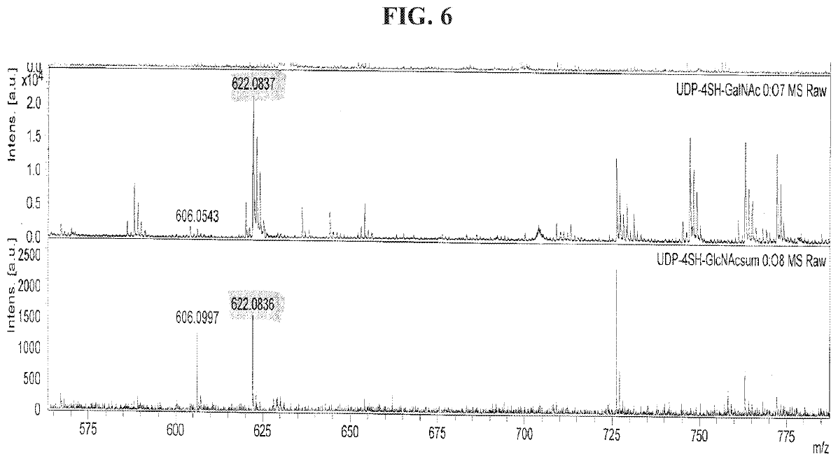

FIG. 6 contains mass spectra depicting the two sulfur-containing UDP-sugars produced in accordance with the schema of FIG. 5 or similar reactions. The top panel indicates the production of UDP-4S-GalNAc, the lower panel indicates the production of UDP-4S-GlcNAc. The masses of the targets are highlighted; the masses were as predicted, 622 Da.

### Materials and Methods Relating to Acceptor, Donor, and Enzyme Reagents

All defined HA and HEP oligosaccharides and polysaccharides in this example were gifts from Hyalose, LLC or Caisson Biotech, LLC (both in Oklahoma City, Okla.). UDP-Glc was purchased from MP Biomedicals (Santa Ana, Calif.). UDP-GlcNAc, UDP-GlcA, and all other chemicals, unless noted, were purchased from Sigma-Aldrich (St. Louis, Mo.). Samples may be further purified as for normal GAGs; for example, by loading onto a Sepharose Q column (GE; Pittsburgh, Pa.; or other anion exchange resins) equilibrated with 50 mM Tris, pH 8.6 (or ammonium acetate or other buffers that keep the GAG in a charged state). The column is then washed with 50 mM buffer (3-6 column volumes, CVs), then 50 mM buffer with 150 mM NaCl (3-6 CVs; or other salts used for ion exchange processes). The target GAGs will typically elute in fractions with 0.3-1.5 M NaCl or other salt, depending on the polymer size (i.e. larger polymers with more negative groups require higher salt levels to be removed from the column). Ultrafiltration, alcohol precipitation, etc may be used to harvest the GAG from the eluted fractions.

An Escherichia coli-derived recombinant truncated (residues 1-703) version of Pasteurella multocida hyaluronan synthase (PmHAS) was used to make HA products. Several E. coli-derived recombinant maltose-binding protein fusions with the P. multocida HEP synthase enzymes were used for making HEP: these include HEP synthase 1 (PmHS1), HEP synthase 2 (PmHS2), and a chimeric protein of these two enzymes called HEP synthase G (PmHS-G). Basically, the exact GAG synthase or glycosyltransferase or enzyme sequence is not critical as long as the analog and the natural donors are transferred to the nascent GAG chain as desired. Pasteurella enzymes (e.g., based on the prototypes PmHAS, PmHS, PmCS) are shown here, but this invention is not limited to these specific enzymes as a novel area is the sulfur-containing GAGs with free sulfhydryl groups (S-tagged) and/or thioglycosidic S-linkages (S-links). For example, the Escherichia enzymes (e.g., from K4, K5 strains) or from other genera with ^{˜}10-50 amino acid motifs or domains with 40-90% homology to the Pasteurella enzymes are also expected to perform in more or less the same fashion. Such examples include (a) E. coli KfoC, Chlorobium CpCS, Avibacterium paragallinarum ApCS (for chondroitin), or (b) E. coli KfiABC enzymes, Avibacterium paragallinarum heparosan synthase (for heparosan), or (c) Avibacterium paragallinarum hyaluronan synthase or Streptococcus SpHAS, SeHAS or Chorella virus CvHAS (for HA).

The sulfur-containing UDP-sugars were then utilized in chemoenzymatic syntheses with glycosaminoglycan synthases to extend functional acceptors or in reactions without acceptors (i.e. de novo polymerization).

### Materials and Methods Relating to Single Sugar Extension and Polymerization Reactions

In general for single sugar extensions of AMAC-, ANTS-labeled (e.g., any dye or tag to facilitate monitoring and analysis; 50-100 pmoles) or unlabeled (^{˜}1 μg) oligosaccharide acceptors were incubated with a specific UDP-sugar, as noted, at 2 mM, 0.2-2 mg/mL synthase enzyme (type as noted in each experiment), 2-10 mM MnCl₂, and 50 mM HEPES, pH 7.2, for 2-20 h at 30° C. HA and chondroitin synthase reactions also included 1M ethylene glycol as a stabilizer.

For polymerization tests, for example, the various oligosaccharide acceptors were incubated with two types of UDP-sugars at 2 mM as noted under the same conditions as the single sugar extensions above. Radioactive UDP-sugars (e.g., UDP-[³H]GlcA) were used to monitor co-polymerization reactions with the thiosugar-donors separated by various modes of chromatography. The general reaction catalyzed by a GAG synthase in these tests is:

n UDP-4S-HexNAc+n UDP-[³H]GlcA+acceptor-->2n UDP+[4S-HexNAc+UDP-[³H]GlcA]ₙ-acceptor

where HexNAc is GlcNAc, GalNAc, or another sugar or sugar derivative.
Reactions used radioactive UDP-GlcA (0.08 mM final; 0.1 microCi ³H) and the authentic UDP-sugars (a positive control) or their thio-analogs (0.5 mM final) with a GAG synthase (1 mg/ml) in neutral pH buffer (50 mM Hepes, pH 7.2) with divalent cation (1 mM Mn²⁺) at 30° C. for 5.5 hours (note: the HA [PmHAS] and chondroitin [PmCS] synthase reactions also employed the stabilizer 1M ethylene glycol). The reactions were quenched (2% SDS detergent), and analyzed by paper chromatography. The small radioactive UDP-sugar precursors are polymerized by the synthase, resulting in larger products (stay at the origin of the paper; any unused precursor migrates down the paper); such data is shown in Table 3.

Chimeric enzymes that included differing portions of both PmHS1 (GenBank Accession #AAL84702) and PmHS2 (GenBank Accession #AAQ55109) were created and tested for their ability to synthesize polymers from the sulfur-containing UDP-4-S-GlcNAc. As shown below in Table 2, the corresponding numbers describe the original residue numbers of the source parental synthase sequence as noted.

TABLE 2
Chimeric heparosan synthase enzyme structures

| | | |
|---|---|---|
| Chimera A = PmHS1 1-133 | PmHS2 168-651 | |
| Chimera B = PmHS1 1-318 | PmHS2 353-651 | |
| Chimera C = PmHS1 1-467 | PmHS2 502-651 | |
| Chimera D = PmHS2 1-167 | PmHS1 134-617 | |
| Chimera E = PmHS2 1-352 | PmHS1 319-617 | |
| Chimera F = PmHS2 1-501 | PmHS1 468-617 | |
| Chimera G = PmHS2 1-167 | PmHS1 134-318 | PmHS2 353-651 |
| Chimera H = PmHS2 1-352 | PmHS1 319-467 | PmHS2 502-651 |
| Chimera I = PmHS1 1-133 | PmHS2 168-352 | PmHS1 319-617 |
| Chimera J = PmHS1 1-318 | PmHS2 353-501 | PmHS1 468-617 |

TABLE 3
Heparosan-based S-link polymer synthesis: Screening
of Chimeric Enzymes for Efficiency

| | | ³H radioactivity |
|---|---|---|
| GAG synthase | Test UDP-sugar | at the origin (dpm) |
| none | none (background) | 7 |
| PmHS-D | UDP-GlcNAc (+control) | 4,140 |
| PmHS-D | UDP-4S-GlcNAc | 42 |
| PmHS-I | UDP-GlcNAc (+control) | 3,900 |
| PmHS-I | UDP-4S-GlcNAc | 42 |
| PmHS-A | UDP-GlcNAc (+control) | 9,230 |
| PmHS-A | UDP-4S-GlcNAc | |
| PmHS-J | UDP-GlcNAc (+control) | 12,200 |
| PmHS-J | UDP-4S-GlcNAc | 480 * |
| PmHS-G | UDP-GlcNAc (+control) | 8,900 |
| PmHS-G | UDP-4S-GlcNAc | 510 * |
| PmHS-B | UDP-GlcNAc (+control) | 9,020 |
| PmHS-B | UDP-4S-GlcNAc | 800 * |

* represents significant thio-sugar incorporation into polymer.

As shown in Table 3, a series of chimeric enzymes (here, combinations of PmHS1 and PmHS2 heparosan synthase amino acid sequences) were tested for their ability to incorporate the authentic UDP-GlcNAc donor (+control) or the unnatural UDP-4-S-GlcNAc donor using a radiochemical incorporation assay and monitoring co-polymerization with UDP-[³H]GlcA (i.e. radioactive polymer is only formed if BOTH UDP-sugars are employed simultaneously by the synthase). Overall, the Chimeric B construct (PmHS-B) appears to be the best catalyst tested thus far for incorporating 4-S-GlcNAc into a heparosan-like S-linked polymer.

### Example 2—S-Tagged and S-Linked GAG Chemoenzymatic Synthesis

Heparanase is an endo-glucuronidase that cleaves the HS chain in a variety of mammalian tissues by hydrolysis of the glycosidic bonds between GlcA and glucosamine residues in the low sulfated domains. The action of heparanase modulates the functions of HS to bind to a multitude of proteins, including growth factors and their receptors, chemokines, enzymes, and extracellular matrix proteins. In some cases, the uncleaved HS lacks the ability to form a complex with protein effector that is needed to induce signaling activity. For example, basic fibroblast growth factor can be sequestered by HS on the cell surface. However, heparanase expressed by metastatic tumor cells and activated cells of the immune system may release FGF eliciting an indirect neovascular response. Thus, heparanase acts as a regulatory switch mediating the release of specifically tailored saccharide structures within HS with restricted binding specificities. The actions of heparanase are linked to numerous human diseases, such as cancer, diabetes, and Alzheimer's.

Described herein are novel GAGs such as a heparosan analog made with thio-linkages at all or various positions in the sugar chain, replacing the natural O-linked glycosidic bond with S-linked bonds at some desirable positions. Such modifications create non-hydrolysable linkages in a similar way that IPTG (isothiopropyl-galactoside), the non-metabolizable inducer (structurally similar to the natural lactose substrate) of the lac/tac promoter, is employed for continuous stimulation of recombinant protein transcription in biotechnology processes. The S-linked HS chains may thus be used as inhibitors for the HS degrading enzyme. The mode of enzyme inhibition is competitive due to the similarity of the artificial S-linked HEP derivative (sulfated on sugar ring to facilitate recognition by the mammalian enzyme's active site) to the native HS substrates, but the thio-glycosidic bond of the S-linked GAG will not be cleaved in a normal fashion by the enzyme (in analogy to the IPTG case described above).

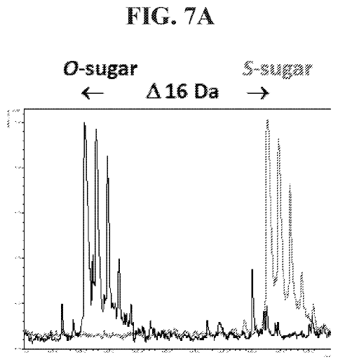

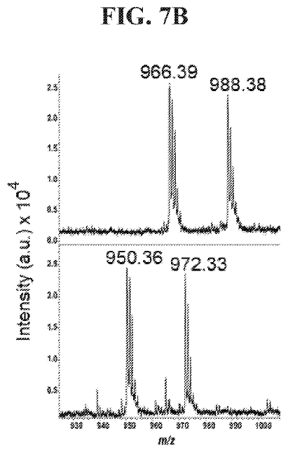

FIGS. 7A and 7B depict non-limiting examples of S-tagged heparosan. The data show mass spectroscopic detection of UDP-4-thio-GlcNAc sugar incorporation by PmHS-G synthase. Tetrasaccharides were made by extension of a HEP-trimer with either natural UDP-GlcNAc (O) or 4-thio-analog (S) as evidenced by the 16 Da mass shift corresponding to the difference in natural abundance sulfur (32 Da) versus oxygen (16 Da) atoms in this MALDI-ToF MS analysis. The 4-thioGlcNAc units from the artificial UDP-sugar donors were thus added to the GAG chain by PmHS-G (a zoomed in perspective is shown in FIG. 7A). FIG. 7B also shows a MALDI-ToF MS analysis of heparosan single sugar extension reactions. PmHS-G added the monosaccharide from either UDP-4S-GlcNAc analog (top) or native UDP-GlcNAc (bottom) to the non-reducing end of a trisaccharide acceptor to form a new tetrasaccharide. The analog species are 16 Da larger than the native species due to the difference in masses of S versus O atoms. (note: both the H+ and the Na+ (+22 Da) forms of the polymers are indicated in the spectra). Using the analog allows the addition of a free sulfhydryl group to the HEP chain resulting in S-tagging or end-labeling.

Typical aqueous enzymatic reaction conditions may be used (e.g., non-limiting examples include neutral pH, 10-100 mM Tris or HEPES buffer, with ^{˜}0.1-20 mM divalent metal cation such as Mn or Mg, temperatures of 10-37° C., etc).

As heparosan is an all ‘4-linked’ polysaccharide, the next sugar added by the enzyme after 4S-GlcNAc (i.e. if UDP-GlcA is also added to the reaction with the thio-sugar analog donor) is joined by a S-linkage rather than the typical O-link found in Nature, in some embodiments. This event is unlike previously created fluoro- and azido-derivatives that were chain terminators (i.e. no repetitive extension or addition of new sugars to the GAG chain) as described in J Org Chem. 2017 Feb. 17; 82(4):2243-2248 and J Org Chem. 2017 Sep. 15; 82(18):9910-9915, respectively, by the laboratories of the inventors.

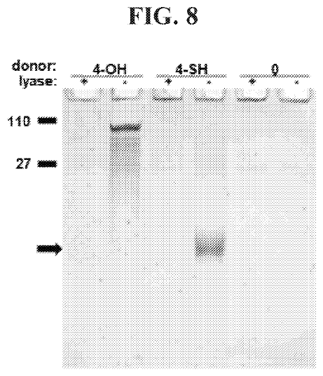

In some embodiments, C—S bonds are resistant to enzymatic digestion. The S-linked polymers can be made in at least 3 types: where only one of the 2 linkages is connected with sulfur (hemi-A or hemi-B, see Table 1 for reference), or where both linkages are so connected (complete S-links, see Table 1). One embodiment that includes a hemi-A polymer has an O-linked bond where the bacterial lyase enzyme cleaves and was digested as predicted between the GlcNAc-1-alpha-4-GlcA sugars (FIG. 8, lyase lanes), but the S-link in appropriately modified HEP polymers would not be degraded by mammalian or human heparanase which cuts at the alternate sugar linkage of the disaccharide unit, between the GlcA-1-beta-4-GlcNAc sugars, thus forming the basis for a competitive inhibitor for cancer treatments.

Polymerization reactions and PAGE analysis. HEP trisaccharide (30 ng/ul) was incubated with 10 mM GlcA and either 10 mM 4-thio- or authentic UDP-GlcNAc, with PmHS-G (0.5 mg/ml), 1 mM MnCl₂, and 50 mM Hepes, pH 7.2, for 20 h at 30° C. The reactions were then diluted in water and split into two aliquots, (i) a untreated control (−) and (ii) a heparin lyase III treated in 50 mM ammonium acetate, pH 7.0, for 20 h at 30° C. (+). The samples were then analyzed on a 6% 1×TBE PAGE (250 V, 15 min) and stained with 0.05% Alcian Blue followed by silver staining (BioRad Silver Stain kit).

The PAGE gel in FIG. 8 shows a PAGE analysis of HEP polymerization reactions, or detection of UDP-4-thio-GlcNAc sugar incorporation into polysaccharides by PmHS-G synthase. Polymerization reactions producing longer HEP chains with either native (4-OH) or analog (4-SH) donors were analyzed for size on PAGE gels and stained. The gel depicts the polysaccharides made with both natural UDP-donors (native HEP) or by using the thio-GlcNAc analog and UDP-GlcA to form Hemi-A HEP.

Both polymers were challenged with bacterial heparin lyase III (+lanes). A portion of the reaction mixture was treated with heparin lyase III (lyase), a specific enzyme that will cut HEP, but not HA, chains; both products are cleaved as seen by the loss of signal in the +lanes. This finding is predicted as the enzyme digests the C—O—C glycosidic link that still exists in both types of HEP species. The data show, among other things, that disaccharide mapping of further modifications (e.g., sulfation of sugar ring) to affirm structure is feasible as the GlcNAc-GlcA bond is O-linked; this mapping is essential for quality control of any HEP-based polymers before use as therapeutics.

Table 4 includes data from a MALDI-ToF MS analysis of native and S-linked HEP. Polymerization reactions with a trimer acceptor (Acc) and UDP-GlcA plus either natural or 4-thio-analog UDP-GlcNAc were performed. The additional mass (16 Da/disaccharide) observed in the Hemi-A HEP in comparison to the natural heparosan indicates that the sulfur group is used in the new glycosidic linkages connecting the GlcA and GlcNAc residues. Predicted masses (Da) are shown in Table 4 on the left of each slash, and observed masses (Da) are shown in Table 4 on the right of each slash. The predicted and observed masses were in good agreement in FIGS. 7, 9, and 10.

TABLE 4

| | | (GlcNAc-GlcA)- | (GlcNAc-GlcA)₂- |
|---|---|---|---|
| Sugar | GlcNAc-Acc | GlcNAc-Acc | GlcNAc-Acc |
| Native HEP | 950.70/950.36 | 1329.81/1329.53 | 1708.92/1708.65 |
| Hemi-A HEP | 966.76/966.39 | 1361.93/1361.38 | 1757.10/1756.49 |

### UDP-4-Thio-Sugars for S-Linked Heparosans

The unnatural donor, UDP-4-thio-GlcNAc, was made for the first time. The observation of S-linked enzymatic coupling in a polysaccharide is an unprecedented finding for any complex carbohydrate. Currently, elsewhere S-links are only found or reported in simple glycosides (e.g., IPTG inducer) or very small carbohydrates such as di- or trisaccharides. The finding with the PmHS catalyst indicates that the thiol group of the transferred sugar served as the attacking nucleophile for the incoming 2^{nd} sugar donor (UDP-GlcA), thus resulting in an S-linked glycosidic or thio-glycosidic bond. Thus a more stable S-linked HEP polysaccharide species can be made for use in the synthesis of a heparanase inhibitor-based therapeutic.

### Acceptors

The acceptor used to start the synchronized polymerization synthesis in some embodiments is a tri- or tetrasaccharide of heparosan (Hep₃ or Hep₄, 3 or 4 sugar units long). The acceptor can be omitted (yields more polydisperse product), or a GlcA-Glc-GlcA HEP trimer can be prepared by stepwise elongation of a synthetic glucuronide primer (e.g., p-nitrophenol-GlcA, NH₂-GlcA, etc.). An important GAG synthase recognition in some embodiments is a pair of GlcA residues for high efficiency reaction priming, thus size-defined species are feasible. If desired, the acceptor's terminal group (e.g., amine or derivative) is available for tagging, conjugation, or immobilization.

### Production of Defined Backbone Polymers

Polysaccharides: Useful polymers include but are not limited to those in the range the ^{˜}5-40 kDa (n=^{˜}12-100). UDP-sugars and a catalyst (such as a recombinant E. coli-derived chimeric promiscuous PmHS-B or -G enzymes) may be added to the trisaccharide acceptor (or other functional acceptor) in reaction buffer (50 mM Tris, pH 7.2, 2 mM MnCl₂; 22° C.) to make a given polymer by synchronized stoichiometrically controlled polymerization (Equation 1). These polymers have a narrow size distribution in the target MW range. In addition, any chemical group on the acceptor will also be present in the polymer; this feature allows tagging, functionalizing, conjugating and/or immobilization of the HS analog as desired.

TABLE 5
Heparosan and some useful heparosan analog targets

| | |
|---|---|
| heparosan (HEP) | (-4-GlcA-1-β-O-4-GlcNAc-1-α-O)ₙ |
| Hemi-A HEP | (-4-GlcA-1-β-S-4-GlcNAc-1-α-O)ₙ |
| HEP with Complete S-links | (-4-GlcA-1-β-S-4-GlcNAc-1-α-S)ₙ |

### Equation 1. Example Polymerization Reaction

n UDP-GlcA+n UDP-GlcNAc+acceptor→2n UDP+[GlcA-GlcNAc]ₙ-acceptor

Notes for Equation 1: Use UDP-4-thiosugar analog for S-linked HEPs; Reaction catalyzed by HEP synthase in presence of divalent cation such as Mg/Mn²⁺

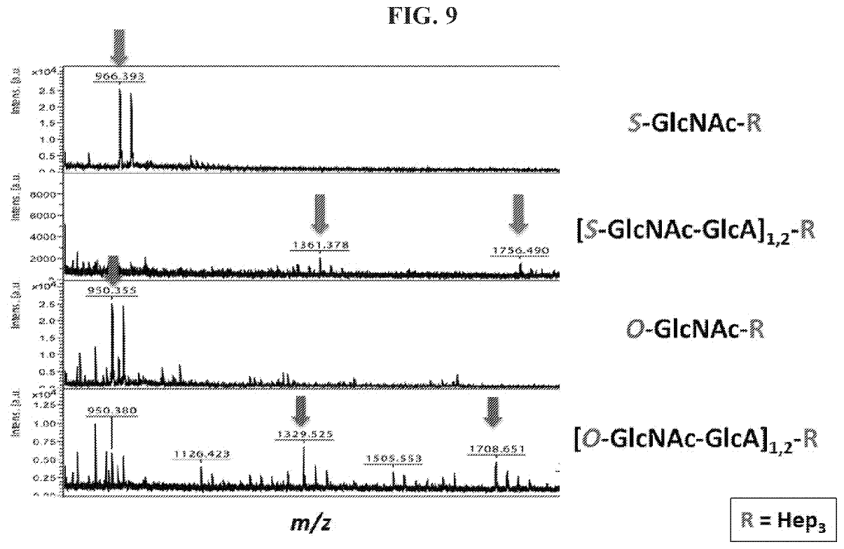

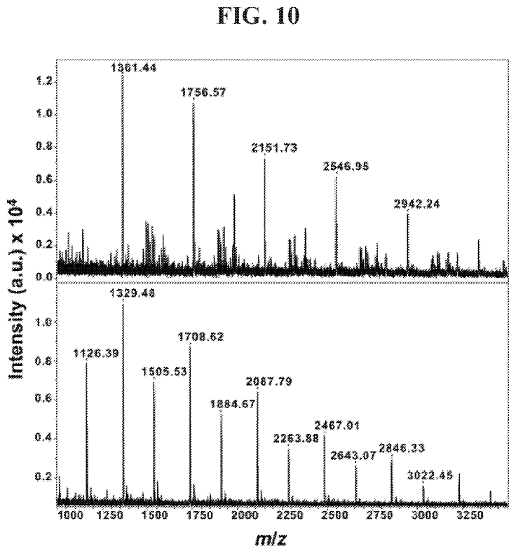

FIG. 9 and FIG. 10 includes MALDI-ToF MS data relating to detection of extension and various polymerization products. Here PmHS-G created polymer chains when incubated with UDP-GlcA and either UDP-4S-GlcNAc analog (FIG. 10, top) or native UDP-GlcNAc (FIG. 10, bottom). The analog allows the creation of C—S—C glycosidic bonds, S-links, instead of the native C—O—C bonds. Therefore, these data also show synthesis of S-tagged and S-linked heparosan.

Reaction conditions may be altered to improve yields (e.g., variables of buffer pH, divalent cation, and additives like salts, alcohols, etc.) as needed. In addition, stabilized enzymes (such as cross-linked enzymes) may be used to increase reaction rates by raising temperatures and/or allow slow reactions to run for the longer time periods needed to accumulate the desired products. Mutant enzymes (e.g., with single point or multiple point changes, deletion or insertion changes, chimeric constructs, or evolved constructs) that better tolerate or favor the thio-donor or their extension products may also be used to improve the sulfur-containing GAG syntheses.

The synthesized polymers may be purified by solvent extraction (to remove enzyme) and ultrafiltration or size exclusion chromatography (SEC; to remove unused UDP-sugars) or anion exchange (AX).

Polymer yields may be checked by carbazole assay for uronic acid. Molecular weights and polydispersity (M_{w}/Mₙ; a measure of the size distribution) may be analyzed by agarose and/or polyacrylamide gel electrophoresis, and multi-angle laser light scattering (HPLC SEC-MALLS). If needed, anion-exchange on Q resin (GE) is used to separate mixtures with charge or size (i.e. longer polymers have more charge so stick tighter) heterogeneity.

The capability to make shorter HS targets is also practical. In general, the reagents and methods for making a series of defined 5- to 12-mers may be similar to the reagents and methods for producing large polysaccharides, except that step-wise elongation may be employed instead of synchronized polymerization. To facilitate controlling the reaction, for example, immobilized single action mutant PmHS-type or PmHAS-type enzymes such as a GlcNAc-transferase or a GlcA-transferase formed by mutating 1 of the 2 DXD motifs of the normally polymerizing synthase may be employed. Synthesis of oligosaccharides may be monitored by mass spectrometry [MS]; upon completion, the next mutant enzyme may be employed to add the next sugar until the target chain is synthesized. In analogy, similar PmHAS-based or related synthases may be used to make shorter HA oligosaccharides as desired.

### Methods for Backbone Modification

HS modification enzymes and modification. Some embodiments include use of an enzyme involved in the biosynthesis of HS to modify a sulfur-containing GAG. Any enzyme involved in biosynthesis of HS (e.g., sulfotransferase, epimerase, etc; as in FIG. 1) may be used. In some embodiments, HS analog backbones are processed in a fashion similar to the HS biosynthetic pathway in mammalian cells. In some embodiments, a particular HS analog polymer is added as input to a reaction with an HS modification enzyme to produce a HS structure that includes a O- and/or N-sulfated (a sulfate or SO3 on the sugar ring hydroxyl or amine moiety, respectively) sulfur-containing heparosan backbone.

Analog product analyses. Disaccharide analyses are performed by completely digesting the GAG polysaccharide with a mixture of heparin lyase 1, 2 and 3 (from various microbes such as Proteus or Flavobacterium) and analyzing the resulting disaccharide mixture by HPLC-ESI-MS. The inventors have found that at least one S-linked analog, the Hemi-A HEP, is sensitive to digestion (FIG. 8), as predicted for a chain with an O-link at the bacterial enzyme cut sites. Oligosaccharide mapping analysis is performed by completely digesting HS polymer with a single heparin lyase to obtain a mixture of oligosaccharides. The oligosaccharide composition is determined by SAX-HPLC with identification based on co-elution with standard oligosaccharides of known structure or by HPLC-ESI-MS. Nitrous acid depolymerization is employed in conjunction with PAGE, HPLC-ESI-MS, MS/MS, and FTICR-EDD-MS.

Heparanase, activity and inhibitor assays. For test purposes, recombinant human heparanase may be expressed in insect cells using baculovirus expression and purified using both heparin-Sepharose and Ni-Sepharose columns. The effect of heparanase on our HS analogs is initially assayed directly by gel analyses; after treatment, sensitive polysaccharides will migrate faster or vanish (tiny oligosaccharides do not typically fix or stain well), while resistant HS analog polymers will remain unchanged. Size exclusion chromatography (SEC) HPLC may be used to more quantitatively monitor the change in chain size upon degradation. Some HS analogs may be bound by heparanase via their substrate-recognition moieties, but polymers with S-linked sugar bonds should be relatively recalcitrant to cleavage and act as competitive inhibitors.

### Health Impact and Utility of S-Linked Heparosan Derivatives

Several uses for defined selective S-linked HS analog molecules are available including inhibitors for treatment of cancer as well as diabetes and Alzheimer's. Such defined carbohydrate-based therapeutics are appealing because the natural sugar structures are biocompatible. In addition, a selective synthetic HS is safer than the existing heterogeneous drug, heparin from porcine intestinal mucosa, with some undesirable activities (e.g., potent anticoagulant) as well as animal origin (potential for adventitious agents e.g., virus or prions) and an insecure supply chain (intentionally contaminated heparin from China associated with ^{˜}100 deaths in the US in 2007-08).

### Example 3—S-Tagged Hyaluronic Acid (HA) Chains

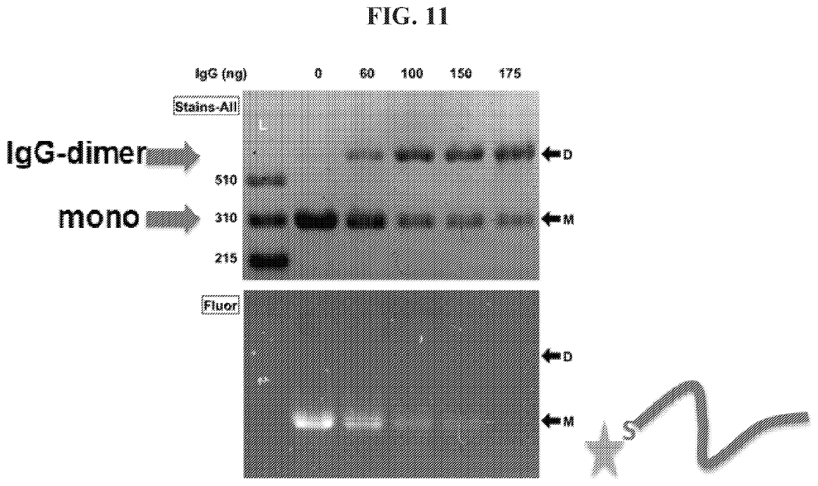

In some embodiments, the sulfur-containing GAG comprises HA. For example, FIG. 11 includes a 300-kDa quasi-monodisperse HA chain that was end-tagged with thio-donor (4S-GlcNAc) at the non-reducing terminus, purified and then treated with fluorescein-maleimide (i.e. a fluorescent reagent that reacts with free thiols and can glow). Agarose gel (run in 1×TAE buffer) analysis with UV illumination (Fluor panel) detects the new fluorescent band. When incubated with a titration of anti-fluorescein IgG, a new staining band of twice the size appears corresponding to a complex of 2 HA chains attached to a bivalent IgG (Stains-all panel); in an additional observation, the tightly binding antibody quenches the fluorescence emission. Thus, the migration shift corresponds to HA dimer (600 kDa) formation and the tight Ab binding quenches the fluorescence. The complexes are destroyed by protease treatment (the IgG is cleaved into small, non-functional peptides) resulting in return of the highly fluorescent HA monomers. These data show synthesis of S-tagged hyaluronan. The new thiol group on the polymers may be used for coupling, conjugating, or immobilizing HA chains.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A composition comprising a polymer, wherein the polymer comprises at least one of the following repeat structures:
a) (-4-GlcA-1-beta-S-4-GlcNAc-1-alpha-)ₙ;
b) S-4-GlcNAc-1-alpha-(-4-GlcA-1-beta-O-4-GlcNAc-1-alpha-)ₙ;
c) (-4-GlcA-1-beta-O-3-GlcNAc(4S)-1-beta-)ₙ;
d) GlcNAc(4S)-1-beta-(-4-GlcA-1-beta-O-3-GlcNAc-1-beta-)ₙ,
wherein n is a positive integer greater than or equal to 1;

2. (canceled)

3. The composition of claim 1, wherein n is greater than or equal to 10.

4. (canceled)

5. The composition of claim 1, wherein n is greater than or equal to 50.

6. (canceled)

7. The composition of claim 1, wherein the polymer is substantially monodisperse in size whereby the polymer has a polydispersity value in a range of from about 1.0 to about 1.5.

8. A method of chemoenzymatically producing a glycosaminoglycan polymer having a repeat structure comprising at least one sulfur-containing sugar unit, the method comprising the steps of:
combining a recombinant glycosaminoglycan synthase or glycosyltransferase with at least one of the following:
a) two UDP-sugars, wherein at least one of the UDP-sugars is a UDP-sulfur-containing sugar;
b) a functional acceptor and at least two UDP-sugars, wherein at least one of the UDP-sugars is a UDP-sulfur-containing sugar;
c) a functional acceptor and one UDP-sulfur-containing sugar;
whereby the recombinant glycosaminoglycan synthase polymerizes the at least two sugars to provide a glycosaminoglycan polymer having a repeat structure, wherein the repeat structure comprises at least one sulfur-containing sugar.

9. The method of claim 8, wherein the UDP-sulfur-containing sugar is 4SH-GlcNAc or 4SH-GalNAc.

10. The method of claim 8, wherein the recombinant glycosaminoglycan synthase or glycosyltransferase is selected from the group consisting of a hyaluronan synthase, a chondroitin synthase, and a heparosan synthase or enzymes possessing the ability to extend a glycosaminoglycan chain.

11. The method of claim 8, wherein the UDP-sulfur-containing sugar is provided in a stoichiometric ratio to the at least one functional acceptor such that the recombinant glycosaminoglycan synthase or glycosyltransferase elongates the at least one functional acceptor to provide the glycosaminoglycan polymer, wherein the glycosaminoglycan polymer is substantially monodisperse in size and has a polydispersity value in a range of from about 1.0 to about 1.5.

12. (canceled)

13. (canceled)

14. (canceled)

15. (canceled)

16. (canceled)

17. (canceled)

18. (canceled)

19. (canceled)

20. The method of claim 1, wherein the functional acceptor comprises a therapeutic agent, and wherein the method is further defined as a method of chemoenzymatically producing a glycosaminoglycan polymer-therapeutic agent conjugate.

21. A bioadhesive composition, comprising at least one of:
the composition of claim 1;
a glycosaminoglycan polymer produced by the method of claim 8; and
the glycosaminoglycan polymer-therapeutic agent conjugate produced by the method of claim 20.

22. A pharmaceutical drug delivery composition, comprising:
at least one conjugate comprising at least one therapeutic drug conjugated to at least one glycosaminoglycan polymer, wherein the glycosaminoglycan polymer is selected from the group consisting of:
(a) the composition of claim 1; and
(b) a glycosaminoglycan polymer produced by the method of claim 8; and
a pharmaceutically acceptable carrier.

23. The pharmaceutical drug delivery composition of claim 22, wherein the pharmaceutical drug delivery composition is a sterile pharmaceutical formulation in a unit dosage format.

24. The pharmaceutical drug delivery composition of claim 22, wherein the at least one therapeutic drug remains active following conjugation to the at least one glycosaminoglycan polymer.

25. The pharmaceutical drug delivery composition of claim 22, wherein the at least one therapeutic drug does not increase immunoreactivity of the at least one glycosaminoglycan polymer.

26. The pharmaceutical drug delivery composition of claim 22, wherein the at least one therapeutic drug is not an adjuvant.

27. The pharmaceutical drug delivery composition of claim 22, wherein each conjugate comprises a single glycosaminoglycan polymer conjugated to the at least one therapeutic drug.

28. The pharmaceutical drug delivery composition of claim 22, wherein each conjugate comprises a plurality of glycosaminoglycan polymers conjugated to the at least one therapeutic drug.

29. The pharmaceutical drug delivery composition of claim 22, wherein the at least one glycosaminoglycan polymer and the at least one therapeutic drug are covalently conjugated.

30. The pharmaceutical drug delivery composition of claim 22, wherein the at least one glycosaminoglycan polymer and the at least one therapeutic drug are non-covalently conjugated.

31. The pharmaceutical drug delivery composition of claim 22, wherein the at least one therapeutic drug is selected from the group consisting of a chemotherapy agent, an antineoplastic agent, a steroid, an antibiotic, an anti-inflammatory agent, an agent that has an action on a central nervous system of the mammalian patient, an antihistaminic, an antiallergic agent, an antipyretic, a respiratory agent, an antimicrobial agent, an antihypertensive agent, a calcium antagonist, an antipsychotic, an agent for Parkinson's disease, a vitamin, an antitumor agent, a cholinergic agonist, a mydriatic, an antidepressant agent, an antidiabetic drug, an anorectic agent, an antimalarial agent, a polypeptide therapeutic, a cytokine, a hormone, an enzyme, an antibody, an antibody fragment, an antiulcerative agent, an anticancer agent, a vaccine antigen, a polynucleotide, a nutrient, a small molecule, and combinations thereof.

32. A method, comprising the step of:
administering a therapeutically effective amount of the pharmaceutical drug delivery composition of claim 22 to a mammalian patient so as to induce a therapeutic effect in the mammalian patient and treat a disease or condition from which the mammalian patient suffers and/or reduce the occurrence of a disease or condition to which the mammalian patient is predisposed.

33. (canceled)

34. (canceled)

35. (canceled)

36. (canceled)
